(19) United States
(12) Patent Application Publication
(10) Pub. No.: US 2013/0338529 A1
(43) Pub. Date: Dec. 19, 2013

(54) BIOELECTRIC SIGNAL MEASUREMENT APPARATUS

(71) Applicants: Masayuki Ishijima, Tokyo (JP); Teiji Ukawa, Tokyo (JP)

(72) Inventors: Masayuki Ishijima, Tokyo (JP); Teiji Ukawa, Tokyo (JP)

(73) Assignee: Nihon Kohden Corporation

(21) Appl. No.: 14/001,926

(22) PCT Filed: Jan. 27, 2012
(86) PCT No.: PCT/JP2012/051852

§ 371 Date: Aug. 28, 2013

(30) Foreign Application Priority Data
Feb. 28, 2011 (JP) 2011042935

(51) Int. Cl. A61B 5/04

(52) U.S. Cl. CPC A61B 5/04
USPC 600/547

(57) ABSTRACT

Provided is a bioelectric signal measurement apparatus that obtains an alternate current component and a direct current component of a bioelectric signal. The apparatus includes a plurality of biomedical electrodes, a switch unit, a differential amplification unit, a bioelectric signal extraction unit, and a timing control unit. The plurality of biomedical electrodes are brought into contact with a living body surface and disposed separately from each other. The switch unit short-circuits the biomedical electrodes via a predetermined short-circuit resistance. The differential amplification unit is connected to the biomedical electrodes and amplifies a difference of electric signals therefrom. The bioelectric signal extraction unit extracts a bioelectric signal from an output signal of the differential amplification unit from the release of short-circuit between the biomedical electrodes to the next timing of short-circuiting. The timing control unit controls timings for short-circuiting the biomedical electrodes and releasing the short-circuit therebetween, which are repeated.

### PRIORITY

Priority is claimed as a national stage application, under 35 U.S.C. §371, to PCT/JP2012/051852, filed Jan. 27, 2012, which claims priority to Japanese Application No. 2011-042935, filed Feb. 28, 2011. Each disclosure of the aforementioned priority applications is incorporated herein by reference in its entirety.

### TECHNICAL FIELD

The present invention relates to a bioelectric signal measurement apparatus.

### BACKGROUND ART

From the viewpoint of reducing a burden to the patient, noninvasive measurement of biological information is desired. As a method for noninvasively measuring biological information, a method for measuring a potential generated by a biological part of a subject to be measured through electrodes for biomedical use placed on a living body surface is generally known.

In the biomedical electrodes placed on the living body surface, an electrode potential is generated by an electric double layer in a metal surface of the biomedical electrodes. As a result, in the biomedical electrodes, a sum of the potential generated by the biological part and the electrode potential appears. However, the potential generated by the biological part is commonly small and also a polarization voltage generated between the living body surface and the biomedical electrodes varies depending on a chemical state and others, resulting in an unstable electrode potential of the biomedical electrodes.

As a technique for accurately extracting an electric signal from a living body (hereinafter, referred to as a bioelectric signal) by reducing effects caused by variations of the polarization voltage, a biological signal sampling apparatus of Patent Document 1 listed below is known. It is disclosed that the biological signal sampling apparatus of Patent Document 1 employs an amplifier having a direct current gain of about 1 and an alternate current gain of several tens with respect to a bioelectric signal.

### PRIOR ART DOCUMENT

### Patent Document

[Patent Document 1] Japanese Unexamined Patent Application Publication No. 6-197877

### SUMMARY OF THE INVENTION

### Problem to be Solved by the Invention

However, in the biological signal sampling apparatus of Patent Document 1, an alternate current component of a bioelectric signal is obtained but a problem that a direct current component is not substantially obtained is produced.

Accordingly, the present invention has been made to solve the above-mentioned problem. Therefore, an objective of the present invention is to provide a bioelectric signal measurement apparatus capable of obtaining not only an alternate current component but also a direct current component of a bioelectric signal.

### Means for Solving the Problem

The objective of the present invention is achieved by the following.

The bioelectric signal measurement apparatus of the present invention includes a plurality of biomedical electrodes, switch unit, differential amplification unit, bioelectric signal extraction unit, and timing control unit.

The plurality of biomedical electrodes are brought into contact with a living body surface and disposed separately from each other. The switch unit short-circuits the biomedical electrodes via a predetermined short-circuit resistor. The differential amplification unit is connected to the biomedical electrodes and amplifies a difference of electric signals from the biomedical electrodes. The bioelectric signal extraction unit extracts a bioelectric signal from an output signal of the differential amplification unit during a period of time from the release of short-circuit between the biomedical electrodes to the next timing of short-circuiting. The timing control unit controls timings for short-circuiting between the biomedical electrodes and releasing the short-circuit therebetween, which are repeated.

### EFFECTS OF THE INVENTION

According to the present invention, with inhibiting effects of variations in a polarization voltage, it is possible to obtain not only an alternate current component but also a direct current component of a bioelectric signal. As a result, it is possible to detect, from a body surface, an injury potential of excitable cells due to, for example, myocardial ischemia or brain infarction.

### BRIEF DESCRIPTION OF THE DRAWINGS

FIG. 1 is a schematic block diagram for illustrating a configuration of a bioelectric signal measurement apparatus in a first embodiment of the present invention.

FIG. 2A is a plan view illustrating one example of the structure of the biomedical electrodes illustrated in FIG. 1; FIG. 2B is a cross-sectional view along the B-B line of FIG. 2A; and FIG. 2C is a plan view illustrating another example of the structure of the biomedical electrodes illustrated in FIG. 1.

FIG. 3 is an equivalent circuit diagram of a portion from the biomedical electrodes to the differential amplification unit in the bioelectric signal measurement apparatus illustrated in FIG. 1.

FIG. 4A is a waveform chart in which an output signal of the differential amplification unit has been recorded for about 130 seconds from measurement initiation; FIG. 4B is an enlarged chart of a pulse waveform in the vicinity of 36.78 seconds of FIG. 4A; and FIG. 4C is an enlarged chart of a pulse waveform in the vicinity of 127.14 seconds of FIG. 4A.

FIG. 5 is a waveform chart for illustrating regression analysis of a pulse waveform using a monoexponential function.

FIG. 6 is a chart illustrating an output result of the bioelectric signal measurement apparatus of the first embodiment of the present invention.

FIG. 7 is a waveform chart for illustrating regression analysis of a pulse waveform using a dual exponential function.

FIG. 8 is a schematic block diagram for illustrating a configuration of a bioelectric signal measurement apparatus in a third embodiment of the present invention.

FIG. 9A is a chart for illustrating a method for calculating a time constant τ_{G}; and FIG. 9B is a chart for illustrating a method for calculating a time constant τ_{F}.

FIG. 10 is a schematic block diagram for illustrating a configuration of a bioelectric signal measurement apparatus in a fourth embodiment of the present invention.

FIG. 11 is a schematic block diagram for illustrating a configuration of a bioelectric signal measurement apparatus in a fifth embodiment of the present invention.

FIG. 12 is a schematic block diagram for illustrating a configuration of a bioelectric signal measurement apparatus in a sixth embodiment of the present invention.

### MODES FOR CARRYING OUT THE INVENTION

Embodiments of the bioelectric signal measurement apparatus of the present invention will now be described with reference to the accompanying drawings. Note that, in the figures , the same reference signs are used for the same members . Further, the dimensional ratios in the drawings are exaggerated for the sake of illustration and may differ from the actual ones.

### First Embodiment

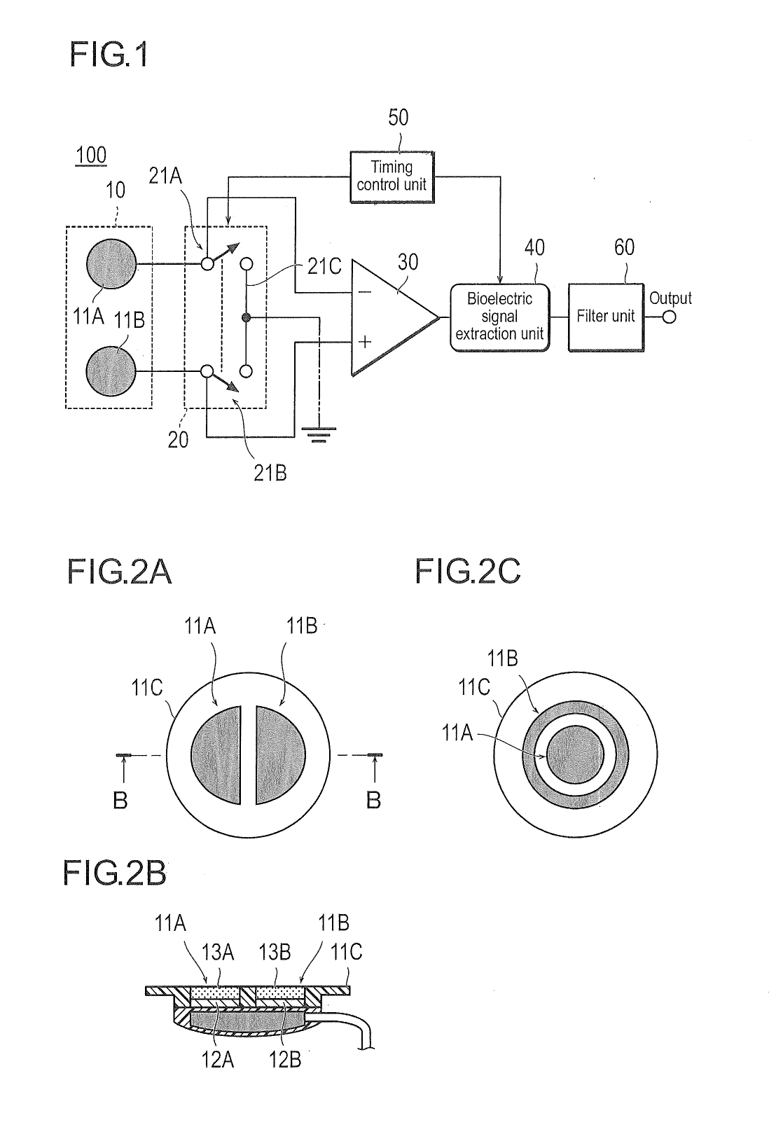

FIG. 1 is a schematic block diagram for illustrating a configuration of a bioelectric signal measurement apparatus in a first embodiment of the present invention. FIG. 2A is a plan view illustrating one example of the structure of the biomedical electrodes illustrated in FIG. 1, FIG. 2B is a cross-sectional view along the B-B line of FIG. 2A, and FIG. 2C is a plan view illustrating another example of the structure of the biomedical electrodes illustrated in FIG. 1.

The bioelectric signal measurement apparatus of the present embodiment repeats short-circuiting between a pair of biomedical electrodes and releasing the short-circuit therebetween at predetermined time intervals to inhibit effects of variations in a polarization voltage, with offsetting a polarization potential difference between the biomedical electrodes.

As illustrated in FIG. 1, a bioelectric signal measurement apparatus 100 of the present embodiment includes a biomedical electrode unit 10, a switch unit 20, a differential amplification unit 30, a bioelectric signal extraction unit 40, a timing control unit 50, and a filter unit 60.

<Biomedical Electrode Unit>

The biomedical electrode unit 10 is placed on a living body surface to lead a potential of the living body surface. The potential of the living body surface is thought to be a potential relevant to a signal source inside the living body (hereinafter, referred to as a biological potential) being superimposed on an electrode potential.

In the present embodiment, the biomedical electrode unit 10 includes a pair of biomedical electrodes 11A and 11B. The biomedical electrodes 11A and 11B are connected to respective input terminals of the switch unit 20 and respective input terminals of the differential amplification unit 30.

As illustrated in FIG. 2A and FIG. 2B, the biomedical electrodes 11A and 11B include electrode elements 12A and 12B and electrically conductive gels 13A and 13B coated on the electrode elements 12A and 12B, respectively. As each of the electrode elements 12A and 12B, a silver/silver chloride electrode element is preferably used, but, for example, a silver electrode element or a carbon electrode element is usable. Further, the electrically conductive gels 13A and 13B exhibit adhesiveness and make contact with a living body surface through an opening of a housing 11C to enhance an electric conductivity between the living body surface and the electrode elements. Note that, if an adhesive is applied to the housing 11C, an electrically conductive gel exhibiting no adhesiveness is employable.

The biomedical electrodes 11A and 11B are accommodated in the housing 11C and juxtaposed separately from each other. In FIG. 2A, faces of the biomedical electrodes 11A and 11B making contact with the living body surface are formed into a semicircular shape and straight line portions of the semicircles are disposed opposite to each other. The biomedical electrode 11A and the biomedical electrode 11B are separated by a part of the housing 11C so as not to make contact with each other. A distance between the biomedical electrode 11A and the biomedical electrode 11B is preferably set to be, for example, several millimeters.

Note that, the shape of the biomedical electrodes 11A and 11B is not limited to a semicircle and may be another shape. For example, in another example of the biomedical electrodes illustrated in FIG. 2C, a face of the biomedical electrode 11A making contact with the living body surface is formed into a circular shape, and a face of the biomedical electrode 11B making contact with the living body surface is formed into a ring shape concentric with the biomedical electrode 11A. Therefore, a pair of the biomedical electrodes 11A and 11B is disposed on respective concentric circles in the housing 11C. In such case, an electrode area of a concentric circle center portion and an electrode area of a periphery are preferably defined to be equal.

Further, the biomedical electrodes 11A and 11B are not necessarily accommodated in one housing, and two independent biomedical electrodes are usable.

<Switch Unit>

The switch unit 20 short-circuits a pair of the biomedical electrodes 11A and 11B. In the present embodiment, the switch unit 20 includes a pair of analog switches 21A and 21B each of which is configured using a FET device.

One terminal of each of the analog switches 21A and 21B is connected to corresponding biomedical electrode 11A or 11B, and the other terminal thereof is connected to a jumper line 21C as a short-circuit resistor. The jumper line 21C can be connected to the ground. Further, a first control signal is input to control terminals of the analog switches 21A and 21B from the timing control unit 50.

The analog switches 21A and 21B are switched on or off in response to the first control signal from the timing control unit 50. When the analog switches 21A and 21B are switched on, the biomedical electrodes 11A and 11B are short-circuited through the jumper line 21C. Thereafter, when the analog switches 21A and 21B are switched off, short-circuit between the biomedical electrodes 11A and 11B is released. Note that, a resistance of the jumper line 21C is very small.

<Differential Amplification Unit>

The differential amplification unit 30 amplifies a difference between electric signals from the biomedical electrodes 11A and 11B and outputs a resulting electric signal. The differential amplification unit 30 includes an instrumentation amplifier configured using, for example, a FET device. One input terminal (inverting input) of the differential amplification unit 30 is connected to the biomedical electrode 11A and the other terminal (non inverting input) thereof is connected to the biomedical electrode 11B. Further, an output terminal of the differential amplification unit 30 is connected to an input terminal of the bioelectric signal extraction unit 40.

When the analog switches 21A and 21B remain on, the biomedical electrodes 11A and 11B are short-circuited, and therefore, an amplitude value of an output signal of the differential amplification unit 30 is substantially 0 [V]. On the other hand, when the analog switches 21A and 21B remain off, electric signals from the biomedical electrodes 11A and 11B are differentially amplified at a predetermined gain to be output to the bioelectric signal extraction unit 40.

<Bioelectric Signal Extraction Unit>

The bioelectric signal extraction unit 40 extracts a bioelectric signal from an output signal of the differential amplification unit 30. An input terminal of the bioelectric signal extraction unit 40 is connected to the output terminal of the differential amplification unit 30, and an output terminal of the bioelectric signal extraction unit 40 is connected to an input terminal of the filter unit 60. Further, a second control signal from the timing control unit 50 is input to a control terminal of the bioelectric signal extraction unit 40.

The bioelectric signal extraction unit 40 includes an A/D converter and a regression analysis unit. The A/D converter converts an analog signal to a digital signal with respect to an output signal of the differential amplification unit 30. The regression analysis unit applies regression analysis, using a monoexponential function, to a pulse waveform of the output signal of the differential amplification unit 30 having been converted to a digital signal.

After receiving the second control signal, the bioelectric signal extraction unit 40 extracts a bioelectric signal from the output signal of the differential amplification unit 30 during a period of time from the release of short-circuit between a pair of the biomedical electrodes 11A and 11B to the next timing of short-circuiting. In the present embodiment, the bioelectric signal is a signal corresponding to a sum of a biological potential and an electrode potential. The thus-extracted bioelectric signal is output to the filter unit 60. Note that, a regression analysis method for a pulse waveform using a monoexponential function will be described later.

In the present embodiment, the regression analysis unit is preferably configured on a platform executing numerical processing at high speed. The regression analysis unit is preferably configured using, for example, FPGA, ASIC, or DSP.

<Timing Control Unit>

The timing control unit 50 controls timings for short-circuiting a pair of the biomedical electrodes 11A and 11B and releasing the short-circuit therebetween, which are to be repeated.

One output terminal of the timing control unit 50 is connected to the control terminals of the analog switches 21A and 21B of the switch unit 20, and the other output terminal of the timing control unit 50 is connected to the control terminal of the bioelectric signal extraction unit 40.

The timing control unit 50 generates the first control signal and transmits the first control signal to the switch unit 20. The first control signal are switched between a high level and a low level so that the analog switches 21A and 21B of the switch unit 20 are repeatedly switched on/off in a predetermined period. Note that, the predetermined period is about 1 to 5 [ms]. The first control signal is generated using, for example, a synchronizing pulse generator.

Further, the timing control unit 50 is also capable of controlling a timing for extracting a bioelectric signal by the bioelectric signal extraction unit 40. In this case, the timing control unit 50 generates the second control signal and transmits the second control signal to the bioelectric signal extraction unit 40. The second control signal is a signal delayed from the first control signal by a predetermined time t_{d}.

<Filter Unit>

The filter unit 60 eliminates a high frequency component from an output signal of the bioelectric signal extraction unit 40 and outputs a resulting signal to an output terminal of the bioelectric signal measurement apparatus 100. The filter unit 60 includes a lowpass filter, for example, having a cutoff frequency of 100 to 1000 [Hz]. The input terminal of the filter unit 60 is connected to the output terminal of the bioelectric signal extraction unit 40, and the output terminal of the filter unit 60 is connected to the output terminal of the bioelectric signal measurement apparatus 100.

The bioelectric signal extraction unit 40 outputs a pulse signal generated by ON/OFF of the analog switches 21A and 21B. The filter unit 60 eliminates a high frequency component of an output signal of the bioelectric signal extraction unit 40 to extract a bioelectric signal which is a continuous analog signal.

The bioelectric signal measurement apparatus 100 of the present embodiment configured as described above includes a pair of the biomedical electrodes 11A and 11B, the switch unit 20, the differential amplification unit 30, the bioelectric signal extraction unit 40, the timing control unit 50, and the filter unit 60. The pair of the biomedical electrodes 11A and 11B are brought into contact with a living body surface and disposed separately from each other. The switch unit 20 short-circuits the biomedical electrodes 11A and 11B. The differential amplification unit 30 is connected to the biomedical electrodes 11A and 11B and amplifies a difference between the electric signals from the biomedical electrodes 11A and 11B. The bioelectric signal extraction unit 40 extracts a bioelectric signal from an output signal of the differential amplification unit 30 during a period of time from the release of short-circuit between the biomedical electrodes 11A and 11B to the next timing of short-circuiting. The timing control unit 50 controls timings for short-circuiting the biomedical electrodes 11A and 11B and releasing the short-circuit therebetween, which are to be repeated.

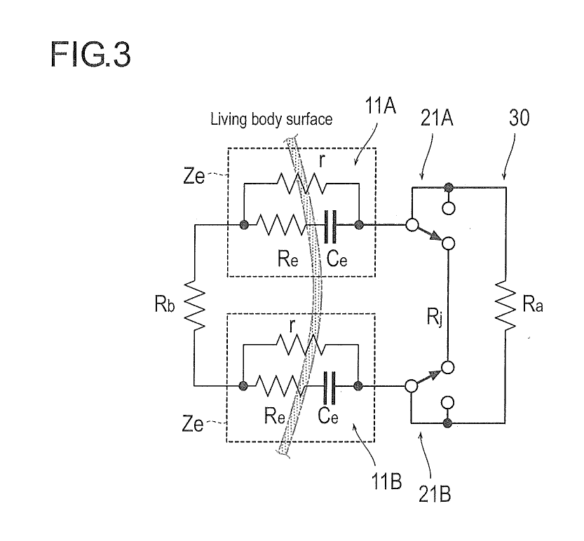

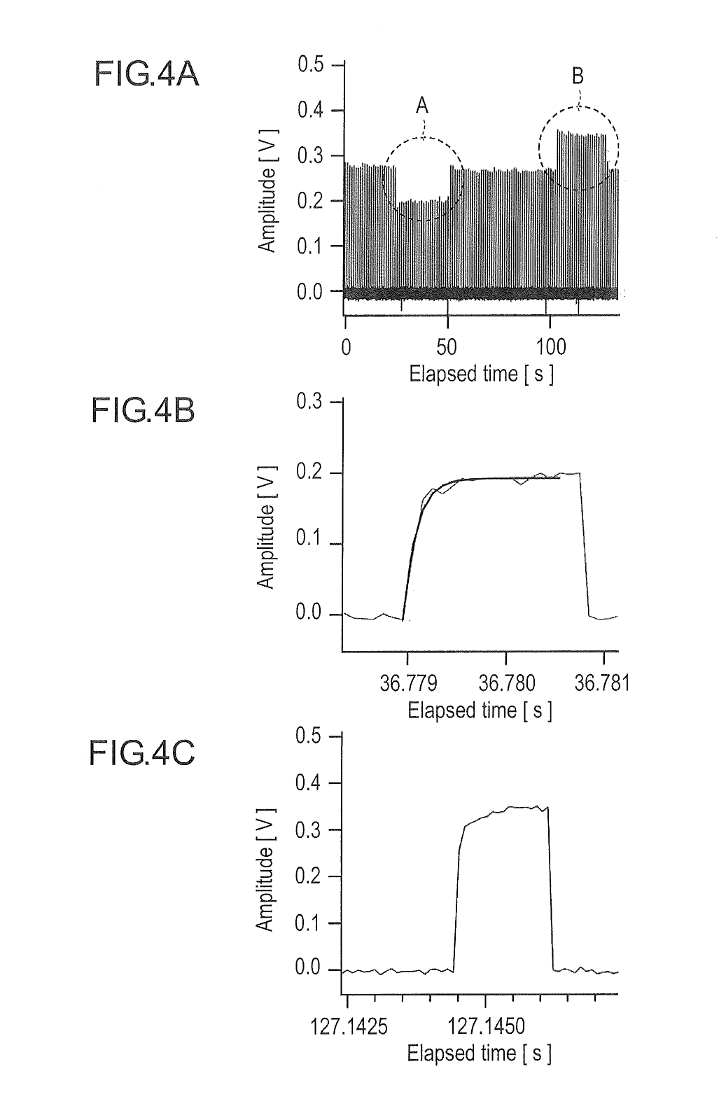

An operation of the bioelectric signal measurement apparatus of the present embodiment will be described below with reference to FIG. 3 and FIG. 4. FIG. 3 is an equivalent circuit diagram of a portion from the biomedical electrodes to the differential amplification unit in the bioelectric signal measurement apparatus illustrated in FIG. 1.

As illustrated in FIG. 3, the equivalent circuit has an impedance Ze for each of the biomedical electrodes 11A and 11B placed on a living body surface, a body resistance Rb, and an input resistance Ra of the differential amplification unit 30.

The impedance Ze is modeled as a configuration where a resistance Re and a capacitance Ce connected in series are connected to a resistance r in parallel. Note that, the capacitance Ce is an electric capacitance between the living body surface and each biomedical electrode and mainly an electric double layer capacitance resulting from an electric double layer in the biomedical electrode surface. The resistance Re and the resistance r represent an electric resistance of each biomedical electrode, including an electric resistance between the living body surface and each biomedical electrode.

The body resistance Rb is an electric resistance of a living body between the biomedical electrodes 11A and 11B and increases in proportion to a distance between the biomedical electrodes 11A and 11B. Further, the input resistance Ra of the differential amplification unit 30 is a large resistance of at least several MΩ.

In FIG. 3, after sufficient time have elapsed from switching off the analog switches 21A and 21B, a polarization potential resulting from a polarization voltage generated between the living body surface and each of the biomedical electrodes 11A and 11B is in equilibrium. The polarization potentials for the biomedical electrodes 11A and 11B are slightly different from each other, depending on a current from a signal source inside a living body, a bias current of the differential amplification unit 30, and a difference of chemical states between the living body surface and the biomedical electrodes.

When the analog switches 21A and 21B are switched on, a pair of the biomedical electrodes 11A and 11B are short-circuited, resulting in being equipotential. As a result, a polarization potential difference between the biomedical electrodes 11A and 11B is canceled. More specifically, when the pair of the biomedical electrodes 11A and 11B are short-circuited, a charge stored in the capacitance Ce is discharged through the resistance Re of the biomedical electrode and the body resistance Rb. Therefore, the potential difference between the biomedical electrodes 11A and 11B decreases. However, since the polarities of the polarization potentials of the biomedical electrodes 11A and 11B are in the same direction, most of the polarization potentials are considered to remain. Note that, such potential change resulting from short-circuit between the biomedical electrodes 11A and 11B is unable to be picked up from the output terminal of the differential amplification unit 30 due to short-circuit of the input of the differential amplification unit 30.

Subsequently, when the analog switches 21A and 21B are switched off after a certain elapsed time, short-circuit between the pair of the biomedical electrodes 11A and 11B is released and then a charge starts to be stored in the capacitance Ce from the signal source inside the living body. At the same time, the polarization potential difference between the biomedical electrodes 11A and 11B also starts to return. This polarization potential difference appears in the output terminal of the differential amplification unit 30 as a pulse waveform having an exponential rise.

A maximum value of the pulse waveform is an overall potential obtained by totaling a biological potential and an electrode potential (hereinafter, referred to as an overall potential). Then, when the analog switches 21A and 21B are allowed to remain off until the pulse wave reaches the maximum value, the polarization potential comes to equilibrium. Since the polarization potential in equilibrium is unstable, a fluctuation of polarization (drift) arises.

FIG. 4A to FIG. 4C are waveform charts each illustrating an output waveform of the differential amplification unit 30 when short-circuiting the biomedical electrodes 11A and 11B and releasing the short-circuit therebetween are repeated. In FIG. 4A to FIG. 4C, the vertical axis represents amplitude [V] and the horizontal axis represents elapsed time [s] from measurement initiation.

FIG. 4A is a waveform chart in which an output signal of the differential amplification unit 30 has been recorded for about 130 seconds from measurement initiation. In FIG. 4A, the biological potential largely changes in the portions represented by A and B. Further, FIG. 4B is an enlarged chart of the pulse waveform in the vicinity of 36.78 seconds of FIG. 4A, and FIG. 4C is an enlarged chart of the pulse waveform in the vicinity of 127.14 seconds of FIG. 4A. As illustrated in FIG. 4B and FIG. 4C, the rise edge of the pulse waveform of the output signal of the differential amplification unit 30 exponentially changes.

Therefore, in the present embodiment, a maximum value of a pulse waveform is predicted by regression analysis to reduce effects of fluctuation in a polarization potential on the overall potential to be measured. More specifically, the analog switches 21A and 21B are switched off and short-circuit between the biomedical electrodes 11A and 11B is released, followed by measurement of a pulsed potential, and thereafter, the analog switches 21A and 21B are switched on to short-circuit the biomedical electrodes 11A and 11B.

Since close arrangement of the biomedical electrodes 11A and 11B and reduction of the body resistance Rb make it possible to reduce a time constant of the equivalent circuit illustrated in FIG. 3, the period of a pulse to be measured is able to be reduced.

With reference to FIG. 5, the regression analysis method for a pulse waveform in the present embodiment will be described below. FIG. 5 is a waveform chart for illustrating regression analysis of a pulse waveform using a monoexponential function. In FIG. 5, the vertical axis represents amplitude [V] and the horizontal axis represents elapsed time [s] from measurement initiation. The present embodiment applies regression analysis to a pulse waveform using a monoexponential function. The monoexponential function is a function represented by the following numerical expression (1).

Numerical expression (1)

y=a₁+b₁·(1−exp(−t/τ) (1)

In numerical expression (1), a₁ of the first term represents a compensation value in calculation; and b₁ of the second term represents a sum of a biological potential and an electrode potential, and τ represents a pseudo-time constant in which a time constant for a biological potential to charge an electric capacitance between a living body and each biomedical electrode such as an electric double layer and a time constant until a polarization potential reaches an equilibrium state are totaled.

As illustrated in FIG. 5, a pulse waveform is sampled with a predetermined sampling period. A regression analysis is applied using a monoexponential function based on plurally sampled values 1 which are obtained, and thereby a₁, b₁, and τ in numerical expression (1) are accurately determined and a regression curve 2 is obtained. In the present embodiment, the sampling period can be set to 0.1 to 0.02 [ms] (sampling speed: 10 to 50 [kHz]). Note that, to prevent from drift contamination, before the polarization potential reaches an equilibrium state, the sampling of a pulsed potential is terminated.

Since being a sum of a biological potential and an electrode potential, b₁ of the second term of numerical expression (1) represents a relative potential with respect to the biological potential. Therefore, b₁ of the second term of numerical expression (1) does not represent a true value of the biological potential. However, when the electrode potential is constant, even the relative potential correlates with the biological potential, and therefore regression analysis using a monoexponential function is effective.

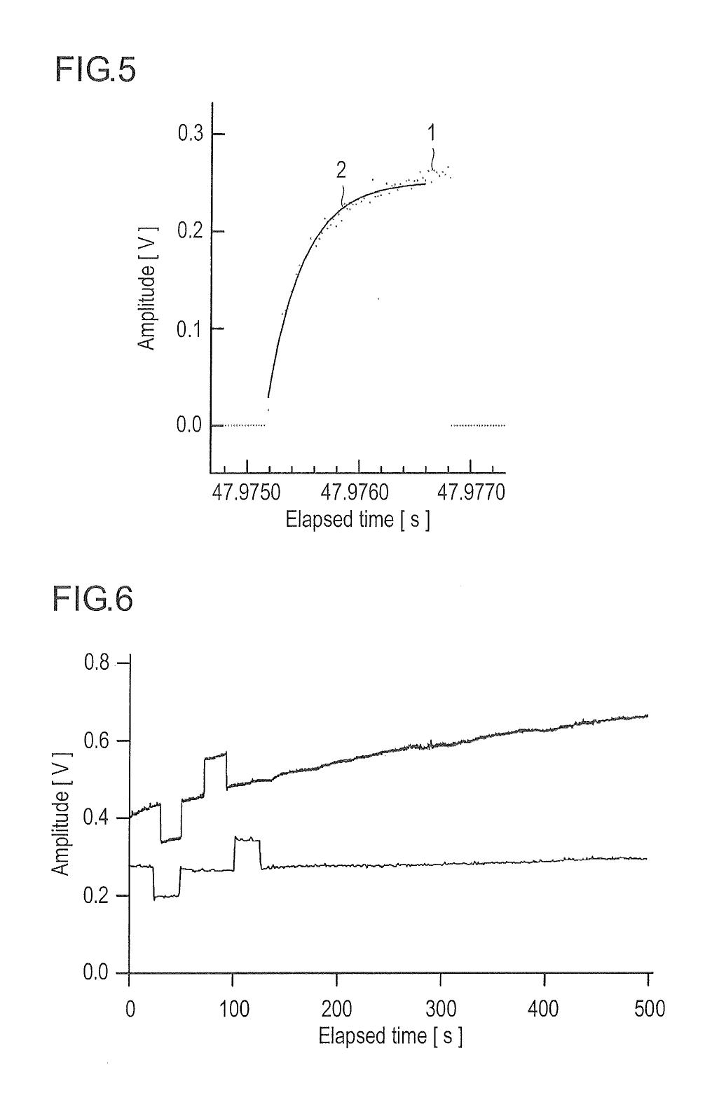

As a result of regression analysis of a pulse waveform using a monoexponential function, a signal corresponding to the sum of the biological potential and the electrode potential is output from the bioelectric signal extraction unit 40 as a bioelectric signal. Since the output signal from the bioelectric signal extraction unit 40 has a continuous pulse waveform in which an amplitude value changes with the magnitude of the sum of the biological potential and the electrode potential, a high frequency component is cut off by a lowpass filter of the filter unit 60 and then the bioelectric signal that is a continuous analog signal is output. With reference to FIG. 6, an example of the bioelectric signal measurement apparatus of the present embodiment will be described below.

### Example

FIG. 6 is a chart illustrating an output result of the bioelectric signal measurement apparatus of the present embodiment. In FIG. 6, the vertical axis represents amplitude [V] and the horizontal axis represents elapsed time [s] from measurement initiation. The upper trace represents an electrooculogram measured directly from biomedical electrodes placed on both sides of the eyes and on the body surface of the face. Further, the lower trace represents a potential measured through the bioelectric signal measurement apparatus of the present embodiment using the same biomedical electrodes as in the upper trace.

In either case of the upper and lower traces, a measurement result of a potential is illustrated, in which the eyes were shifted to the left by about 30 degrees after about 25 seconds from measurement initiation and stopped; the eyes were then returned to the front and thereafter shifted to the right by about 30 degrees; and after about 130 seconds (after about 100 seconds in the upper trace), the eye was fixed up to 500 seconds after returning to the front.

As illustrated in FIG. 6, the upper trace changes with a positive slope due to effects of fluctuation in a polarization voltage. On the other hand, the lower trace remains substantially horizontal without effects of fluctuation in the polarization voltage.

As mentioned above, the present embodiment having been described produces the following effects.

(a) In the bioelectric signal measurement apparatus of the present embodiment, short-circuit between a pair of biomedical electrodes placed on a living body surface is released and thereafter sampling of a pulsed potential is terminated before a polarization potential reaches an equilibrium state, and then the pair of the biomedical electrodes are short-circuited. The bioelectric signal extraction unit extracts a bioelectric signal from an output signal of the differential amplification unit during a period of time from the release of short-circuit between the pair of the biomedical electrodes to the next timing of short-circuiting. Therefore, with inhibiting effects of fluctuation in a polarization voltage, not only an alternate current component but also a direct current component of a bioelectric signal are obtained. As a result, it is possible to detect, from a body surface, an injury potential of excitable cells due to, for example, myocardial ischemia or brain infarction having been conventionally able to be measured only by SQUID (Superconducting Quantum Interference Device). Further, no electrode paste needs to be used in order to inhibit effects of fluctuation in a polarization voltage.

(b) In addition, the bioelectric signal measurement apparatus of the present invention is applicable to cases where bioelectric signals are extracted from living organisms such as roots and leaves of plants. Therefore, no special electrodes need to be used to inhibit effects of fluctuation in a polarization voltage.

### Second Embodiment

In the first embodiment, a bioelectric signal has been extracted by regression analysis of a pulse waveform using a monoexponential function. In the second embodiment, a bioelectric signal is extracted by regression analysis of a pulse waveform using a dual exponential function.

The present embodiment has the same configuration as in the first embodiment except the configuration of the bioelectric signal extraction unit 40. Therefore, description of configurations other than the configuration of the bioelectric signal extraction unit 40 will be omitted.

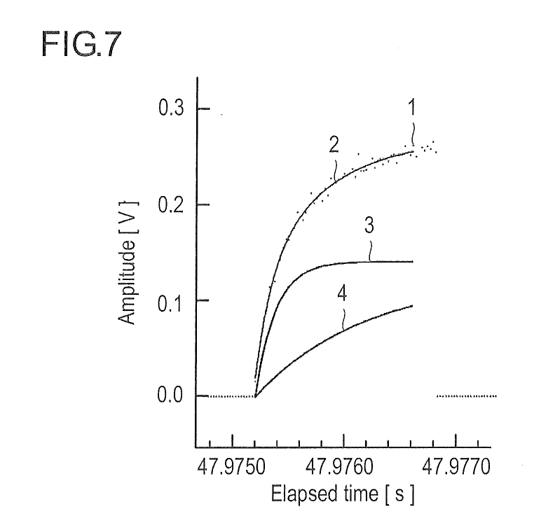

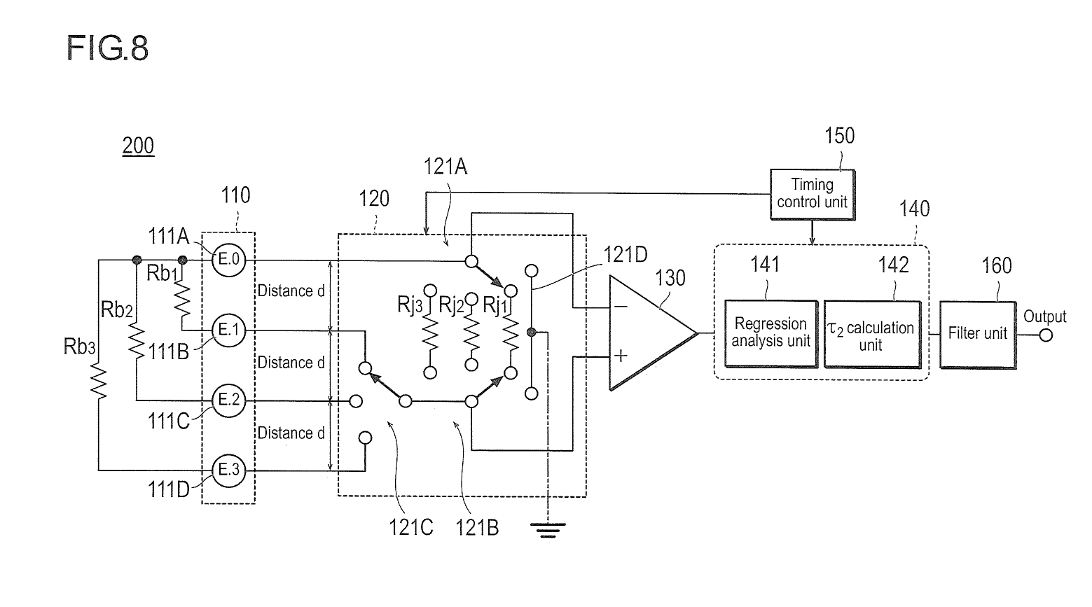

The bioelectric signal extraction unit 40 of the present embodiment applies a dual exponential regression analysis to a pulse waveform output from the differential amplification unit 30 and then extracts a bioelectric signal corresponding to a sum of a biological potential and an electrode potential . With reference to FIG. 7, a regression analysis method for a pulse waveform in the present embodiment will be described below. FIG. 7 is a waveform chart for illustrating regression analysis of a pulse waveform using a double exponential function. In FIG. 7, the vertical axis represents amplitude [V] and the horizontal axis represents elapsed time [s] from measurement initiation. The dual exponential function is a function represented by the following numerical expression (2).

Numerical expression (2)

y=a₂+b₂·(1−exp(−t/τ₁)+c·(1−exp(−t/τ₂)) (2)

In numerical expression (2), a₂ of the first term represents a compensation value in calculation; b₂ of the second term represents a biological potential and τ₁ represents a time constant for the biological potential to charge an electric double layer capacitance; and c of the third term represents the electrode potential and τ₂ represents a time constant until the polarization potential reaches an equilibrium state.

As illustrated in FIG. 7, a pulse waveform is sampled with a predetermined sampling period. To obtain a₂, b₂, c, τ₁, and τ₂, by regression analysis, at least N=5 samples are needed. Regression analysis using obtained sampling values 1 determines a₂, b₂, c, τ₁, and τ₂ in numerical expression (2). In FIG. 7, the curve 2 illustrates a regression curve, the curve 3 illustrates a curve represented by the second term of numerical expression (2), and the curve 4 illustrates a curve represented by the third term of numerical expression (2).

The regression analysis of a pulse waveform using a dual exponential function of the present embodiment enhances accuracy to the extent of falling below 30%, as a maximum, of a chi-square value of regression analysis of a pulse waveform using a monoexponential function.

As mentioned above, the present embodiment having been described produces the following effect in addition to the effects of the first embodiment.

(c) The bioelectric signal extraction unit applies a dual exponential regression analysis to a pulse waveform. As a result, it is possible to accurately calculate a biological potential.

### Third Embodiment

In the second embodiment, a time constant τ₂ has been calculated by regression analysis of a pulse waveform using a dual exponential function. In the third embodiment, prior to regression analysis, an approximate value of a time constant τ₂ is calculated and thereafter this approximate value is applied to the time constant τ₂ of regression analysis.

As described above, execution of regression analysis of a pulse waveform using a dual exponential function determines a₂, b₂, c, τ₁, and τ₂ in numerical expression (2). However, when sampled data contains noise (unexpected external noise, an unstable electrode potential, and others), a calculation result may have an error. Further, the time constant τ₂ may be affected by uncertainties in a contact face between a living body surface and biomedical electrodes.

Therefore, in the present embodiment, as described below, prior to regression analysis, an approximate value of a time constant τ₂ is calculated and thereafter this approximate value is applied to τ₂ of regression analysis to reduce a calculation error in regression analysis.

The time constant τ₂ is considered to be markedly larger than the time constant τ₁ due to accompanying chemical changes. Therefore, if the time constant τ₂ is a value unique to a specific biomedical electrode, the time constant τ₂ is able to be approximately calculated. With reference to FIG. 8, a method for approximately calculating the time constant τ₂ will be described below.

FIG. 8 is a schematic block diagram for illustrating a configuration of a bioelectric signal measurement apparatus in the third embodiment. As illustrated in FIG. 8, a bioelectric signal measurement apparatus 200 of the present embodiment includes a biomedical electrode unit 110, a switch unit 120, a differential amplification unit 130, a bioelectric signal extraction unit 140, a timing control unit 150, and a filter unit 160. Since the configurations of the differential amplification unit 130 and the filter unit 160 are the same as in the first embodiment, description thereof will be omitted.

<Biomedical Electrode Unit>

The biomedical electrode unit 110 includes four biomedical electrodes 111A to 111D placed on a living body surface. Each of the distances between the biomedical electrodes 111A and 111B, the biomedical electrodes 111B and 111C, and the biomedical electrodes 111C and 111D is d. A body resistance between the biomedical electrodes 111A and 111B is Rb1, a body resistance between the biomedical electrodes 111A and 111C is Rb2=2×Rb1, and a body resistance between the biomedical electrodes 111A and 111D is Rb3=3×Rb1. Since concrete structures of the biomedical electrodes 111A to 111D are the same as the configurations in the first embodiment, detailed description thereof will be omitted.

<Switch Unit>

The switch unit 120 selects a pair of biomedical electrodes from a plurality of biomedical electrodes 111A to 111D. Note that, one of the paired electrodes is the biomedical electrode 111A. Further, the switch unit 120 short-circuits the pair of the biomedical electrodes through a predetermined short-circuit resistor. Note that, the predetermined short-circuit resistor is any one of Rj1 to Rj3 and a jumper line 121D. A resistance of the jumper line 121D is very small.

In the present embodiment, the switch unit 120 includes three analog switches 121A to 121C configured using a FET device. Each of the analog switches 121A and 121B has first to fifth terminals and the first terminal is connected to any one of the second to fifth terminals. Further, the analog switch 121C has first to fourth terminals and the first terminal is connected to any one of the second to fourth terminals.

The first terminal of the analog switch 121A is connected to the biomedical electrode 111A and one input terminal (inverting input) of the differential amplification unit 130, and the second to fifth terminals each are connected to one terminal of the short-circuit resistors Rj1 to Rj3 or the jumper line 121D. On the other hand, the first terminal of the analog switch 121B is connected to the other input terminal (noninverting input) of the differential amplification unit 130, and the second to fifth terminals each are connected to the other terminal of the short-circuit resistors Rj1 to Rj3 or the jumper line 121D. The jumper line 121D is connectable to the ground. Further, the first terminal of the analog switch 121C is connected to the first terminal of the analog switch 121B, and the second to fourth terminals each are connected to the biomedical electrodes 111B to 111D.

Further, first and second control signals are input to the control terminals of the analog switches 121A to 121C from the timing control unit 150.

When receiving the first control signal from the timing control unit 150, the analog switches 121A and 121B switch the connection between the first terminal and the second to fifth terminals to select any one of the short-circuit resistors Rj1 to Rj3 and the jumper line 121D. Further, when receiving the second control signal from the timing control unit 150, the analog switch 121C switches the connection between the first terminal and the second to fourth terminals to select anyone of the biomedical electrodes 111B to 111D. For example, when the analog switches 121A and 121B select the short-circuit resistor Rj1 and also the analog switch 121C selects the biomedical electrode 111B, the biomedical electrodes 111A and 111B are short-circuited through the short-circuit resistor Rj1.

<Bioelectric Signal Extraction Unit>

The bioelectric signal extraction unit 140 includes an A/D converter, a regression analysis unit 141, and a τ₂ calculation unit 142. The A/D converter converts an analog signal to a digital signal with respect to an output signal of the differential amplification unit 130. The regression analysis unit 141 applies a dual exponential or monoexponential regression analysis to a pulse waveform of the output signal of the differential amplification unit 130 having been converted to a digital signal. Then, the τ₂ calculation unit 142 calculates time constants τ_{G} and τ_{F} as approximate values of a time constant τ₂ from the pulse waveform of the output signal of the differential amplification unit 130.

<Timing Control Unit>

The timing control unit 150 controls the switch unit 120 and the bioelectric signal extraction unit 140. In the present embodiment, the timing control unit 150 controls the bioelectric signal extraction unit 140 to calculate the time constants τ_{G} and τ_{F} and then to extract a bioelectric signal.

The timing control unit 150 includes a CPU, a memory, and a synchronizing pulse generator. The CPU controls the bioelectric signal extraction unit 140 in accordance with a program stored on the memory. Specifically, when the biomedical electrodes 111A to 111D are placed on a living body surface and measurement is initiated, then the timing control unit 150 outputs instructions to the bioelectric signal extraction unit 140 so as to calculate the time constants τ_{G} and τ_{F}. Further, the CPU generates a selection order with respect to the short-circuit resistors Rj1 to Rj3 and the biomedical electrodes 111B to 111D. Then, based on a generated selection order, first and second control signals are generated.

The first control signal controls which one of the short-circuit resistors Rj1 to Rj3 and the jumper line 121D the analog switches 121A and 121B select at what timing. The second control signal controls which one of the biomedical electrodes 111B to 111D the analog switch 121C selects at what timing.

The synchronizing pulse generator outputs a pulse signal in which a high level and a low level thereof are switched at predetermined time intervals. A third control signal is generated based on the pulse signal. The third control signal controls a timing for extracting a bioelectric signal by the bioelectric signal extraction unit 140.

After calculation of the time constants τ_{G} and τ_{F}, the timing control unit 150 controls the bioelectric signal extraction unit 140 so as to extract a bioelectric signal. At that time, the analog switches 121A and 121B select the jumper line 121D.

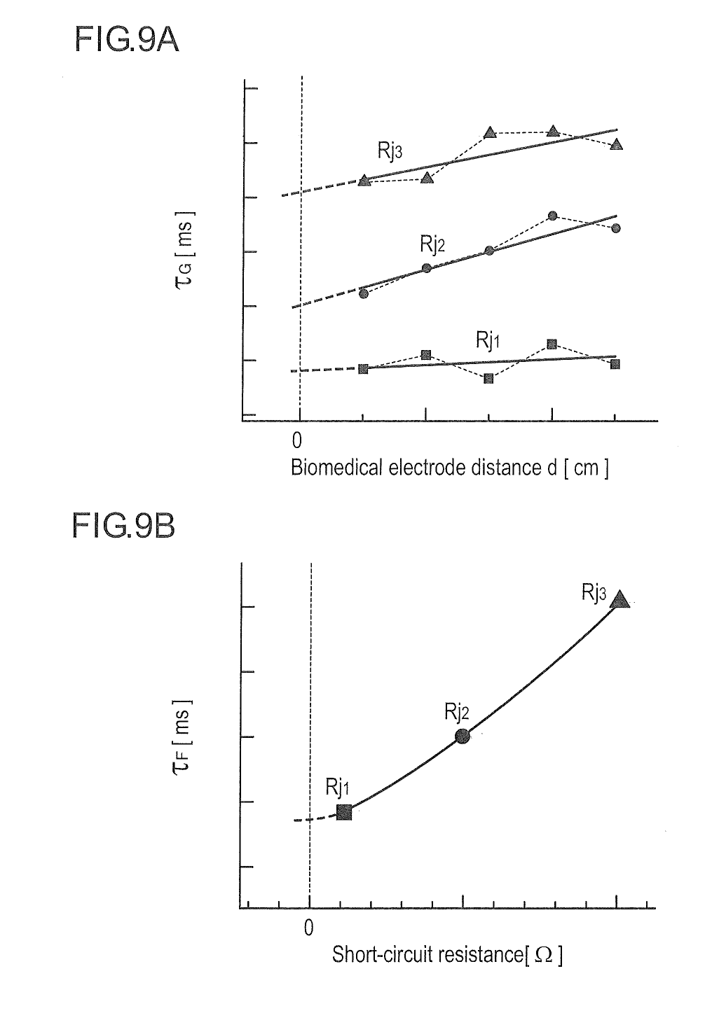

With reference to FIG. 9, a method for calculating the time constants τ_{G} and τ_{F} by the τ₂ calculation unit 142 will be described below. FIG. 9A is a chart for illustrating a method for calculating the time constant τ_{G} and FIG. 9B is a chart for illustrating a method for calculating the time constant τ_{F}. In FIG. 9A, the vertical axis represents time constant τ_{G} [ms] and the horizontal axis represents biomedical electrode distance d [cm] . Further, in FIG. 9B, the vertical axis represents time constant τ_{F} [ms] and the horizontal axis represents short-circuit resistance [Ω].

In FIG. 8, a time constant of an entire closed circuit making a circle through the body resistance Rb, the short-circuit resistance Rj, and the electrode resistance Re is designated as τ′. Further, a time constant associated with the body resistance Rb is designated as τ_{B}; a time constant associated with the short-circuit resistance Rj is designated as τ_{J}; and a time constant associated with a sum of a current induced by a polarization action and an input bias current is designated as τ_{F}. If the above-mentioned definitions are made, the time constant τ′ is represented by the following numerical expression (3).

Numerical expression (3)

τ′=τ_{B}+τ_{J}+τ_{F} (3)

In numerical expression (3) mentioned above, when τ_{B}=τ_{J}=0, τ_{F} can be determined. Procedures for calculating the time constant τ_{G} when τ_{B}=0 and thereafter procedures for determining the time constant τ_{F} when τ_{B}=τ_{J}=0 will be described below.

Initially, the timing control unit 150 controls the switch unit 120 so that the analog switches 21A and 21B select the short-circuit resistor Rj1 and also the analog switch 21C selects the biomedical electrode 111B. As a result, a pulse waveform is output from the differential amplification unit 130. The bioelectric signal extraction unit 140 calculates a fall time constant g(Rb1) by applying regression analysis to a pulse waveform.

In the same manner, the biomedical electrode 111C is selected to calculate a fall time constant g(Rb2) by regression analysis and then the biomedical electrode 111D is selected to calculate a fall time constant g(Rb3) by regression analysis.

Subsequently, as indicated in the following numerical expression (4) and FIG. 9A, calculated time constants g(Rb1) to g(Rb3) are extrapolated and then a time constant τ_{G} when d=0 (Rb=0), that is, τ_{B}=0 is calculated.

Numerical expression (4)

τ_{G}=g(Rb→0) (4)

In the same manner, when changes have been made to the short-circuit resistances Rj2 and Rj3, fall time constants in Rb1 to Rb3 are calculated by regression analysis to calculate τ_{G} for each Rj . Hereinafter, τ_{G} at a short-circuit resistance Rj will be expressed as f(Rj).

As indicated in the following numerical expression (5) and FIG. 9B, calculated time constants f(Rj1) to f(Rj3) are extrapolated and then a time constant τ_{F} when Rb=0 and Rj=0, that is, τ_{B}=0 and τ_{J}=0 is calculated.

Numerical expression (5)

τ_{F}=f(Rj[τ_{G}]→0) (5)

Note that, in extrapolation of the time constant g(Rb) and the time constant f(Rj), a straight line or a function such as a logarithmic function and the like is usable.

Procedures for calculating the time constants τ_{G} and τ_{F} are summarized as follows.

Initially, with respect to a specific short-circuit resistance, a body resistance Rb between a pair of biomedical electrodes is changed and a time constant of a pulse waveform output from the differential amplification unit 130 is extrapolated to calculate a time constant τ_{G} when the body resistance Rb is 0. Then, a short-circuit resistance Rj is changed and the above processing is carried out, followed by extrapolation of each time constant τ_{G} to calculate a time constant τ_{F} when the short-circuit resistance Rj is 0.

The time constant τ_{F} is a time constant determined by a sum of a current induced by a polarization action and an input bias current. To separate these two time constants, in the same manner as the method for calculating the time constants τ_{G} and τ_{F} as described above, a differential amplification unit having different bias current is used and a time constant is calculated. Then, the results are extrapolated to calculate a time constant τ₂ unique to biomedical electrodes when an input bias current=0. Note that, when a bias current is small, the time constant due to the input bias current is negligible.

As mentioned above, the method for calculating the time constants τ_{G} and τ_{F} as approximate values of the time constant τ₂ has been described. In the present embodiment, a calculated time constant τ_{G} or τ_{F} is applied to τ₂ in regression analysis of a pulse waveform using a double exponential function. Therefore, since τ₂ of numerical expression (2) is determined prior to regression analysis, the accuracy of regression analysis of the time constant τ₁ and the biological potential τ₂ is able to be enhanced.

The biological potential b₂ affects a value of the time constant τ₂. Therefore, a calculated approximate value of the time constant τ₂ may deviate slightly from the real value. However, since the magnitude of the polarization potential in the overall potential is much larger than the magnitude of the biological potential b₂ (estimated to be at least 500 times), an effect of the biological potential b₂ on the value of the time constant τ₂ is thought to fall within an acceptable error.

Further, as described above, in the present embodiment, using (i) four biomedical electrodes 111A to 111D placed on a living body surface and (ii) three short-circuit resistors Rj1 to Rj3, a time constant in each combination of the biomedical electrodes and the short-circuit resistances is calculated. Then, calculated time constants are extrapolated and then time constants τ_{G} and τ_{F} are calculated as approximate values of the time constant τ₂ to calibrate a measurement system. A calculated time constant τ_{G} or τ_{F} is applied to τ₂ in regression analysis of a pulse waveform using a double exponential function and a targeted biological potential is calculated.

However, when a personal difference of electrophysiological values such as “an impedance inside a living body” falls within an acceptable error, it is possible to carry out measurement using only two (a pair of) biomedical electrodes 111A and 111B without two biomedical electrodes 111C and 111D of the four biomedical electrodes of above (i). As a result, biological potential measurement becomes remarkably easier.

On the other hand, with respect to the three short-circuit resistors of above (ii), since the case where there is a personal difference in electrophysiological values such as “a contact impedance on the skin surface” results in a decrease in measurement accuracy, it is not preferable to reduce the number of short-circuit resistors, but when electrodes and others that have no personal difference are used, it is possible to reduce the number of short-circuit resistors.

For example, in the following two cases, it is possible to calculate the time constant τ_{G} or τ_{F} using two (a pair of) biomedical electrodes 111A and 111B.

Firstly, in the case where a body resistance Rb per unit distance is known, an inclination in each short-circuit resistance Rji illustrated in FIG. 9A is also known. Therefore, if a time constant τ_{G} is measured in one known biomedical electrode distance, it is possible to calculate a time constant τ_{G} in a short-circuit resistance Rji when the body resistance Rb is 0.

In other words, when a body resistance per unit distance is known, with respect to a specific short-circuit resistance, a pair of biomedical electrodes are disposed separately with a predetermined distance and then a time constant of a pulse waveform output from the differential amplification unit 130 is measured to calculate a time constant τ_{G} when the above body resistance is 0. This processing is carried out with changes in the short-circuit resistance and the time constant τ_{G} for each short-circuit resistance is extrapolated. Thereby, a time constant τ_{F} when the short-circuit resistance is 0 is calculated and then the τ_{F} is applied to τ₂.

Secondly, when the biomedical electrode distance is small, Rb is negligible. In this case, a time constant τ_{G} measured in each short-circuit resistance Rji can be considered a time constant τ_{G} when the body resistance Rb is 0, and then information on the biomedical electrode distance is unnecessary.

In other words, with respect to a specific short-circuit resistance, when a pair of biomedical electrodes are disposed close to each other with making no contact, a time constant of a pulse waveform output from the differential amplification unit 130 is measured and a time constant when the body resistance is 0 is designated as τ_{G}. This processing is carried out with changes in the short-circuit resistance and the time constant τ_{G} for each short-circuit resistance is extrapolated. Thereby, a time constant τ_{F} when the short-circuit resistance is 0 is calculated and then the τ_{F} is applied to τ₂.

Note that, for more accurate calculation, information on blood chemical components and the like obtained by testing prior to measurement initiation of a biological potential using the bioelectric signal measurement apparatus 100 also makes it possible to estimate a value of a personal body resistance Rb.

### Example

Electrocardiogram electrode Vitrode-L (produced by Nihon Kohden Corp.) was used as biomedical electrodes, and an amplifier MEG6116 (produced by Nihon Kohden Corp.) for medical use was used as a differential amplification unit to calculate τ_{F}. As a result, τ_{F} was about 2 [ms].

As mentioned above, the present embodiment having been described produces the following effect in addition to the effects of the first and second embodiments.

(d) The bioelectric signal measurement apparatus of the present embodiment determines a value of the time constant τ₂ and thereafter carries out regression analysis of a pulse waveform using a dual exponential function. As a result, the accuracy of regression analysis of the time constant τ₁ and the biological potential b₂ is able to be enhanced.

### Fourth Embodiment

In the first to third embodiments, a pulse waveform is analyzed by regression analysis using an exponential function to inhibit effects of fluctuation in a polarization voltage. In the fourth embodiment, an amplitude value of a pulse waveform is maintained for a period of time sufficiently shorter than a time constant τ₂ until a polarization potential reaches an equilibrium state to inhibit effects of fluctuation in a polarization voltage.

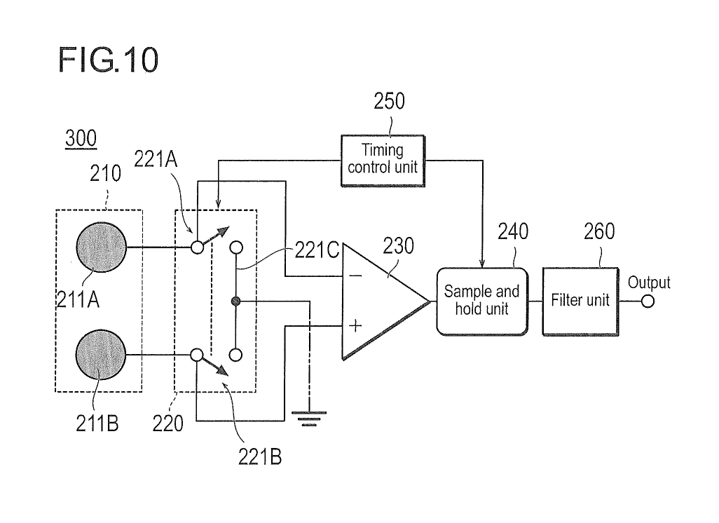

FIG. 10 is a schematic block diagram for illustrating a configuration of a bioelectric signal measurement apparatus of the fourth embodiment. As illustrated in FIG. 10, a bioelectric signal measurement apparatus 300 of the present embodiment includes a biomedical electrode unit 210, a switch unit 220, a differential amplification unit 230, a sample and hold unit 240, a timing control unit 250, and a filter unit 260. In the present embodiment, configurations except for the sample and hold unit 240 are the same as in the first embodiment and therefore description of the above configurations will be omitted.

<Sample and Hold Unit>

The sample and hold unit 240 extracts a bioelectric signal from an output signal of the differential amplification unit 230. An input terminal of the sample and hold unit 240 is connected to an output terminal of the differential amplification unit 230, and an output terminal of the sample and hold unit 240 is connected to an input terminal of the filter unit 260. Further, the second control signal is input to a control terminal of the sample and hold unit 240 from the timing control unit 250.

The sample and hold unit 240 holds an amplitude value of a pulse waveform for a period of time sufficiently shorter than a time constant τ₂ until a polarization potential reaches an equilibrium state during a period of time from the release of short-circuit between a pair of biomedical electrodes 211A and 211B to the next timing of short-circuiting in response to the second control signal.

As illustrated in FIG. 7, the time constant τ₂ until a polarization potential reaches an equilibrium state is markedly larger than the time constant τ₁ for a biological potential to charge an electric capacitance between a living body and each biomedical electrode. Therefore, when a timing for holding a pulse waveform is set to be a period of time sufficiently shorter than τ₂, effects of fluctuation in a polarization voltage is able to be inhibited.

As mentioned above, the present embodiment having been described produces the following effect in addition to the effects of the first to third embodiments.

(e) The sample and hold unit holds an amplitude value of a pulse waveform for a period of time sufficiently shorter than the time constant τ₂ until a polarization potential reaches an equilibrium state. Therefore, with inhibition of effects of fluctuation in a polarization voltage, a bioelectric signal is able to be extracted using a simple configuration.

### Fifth Embodiment

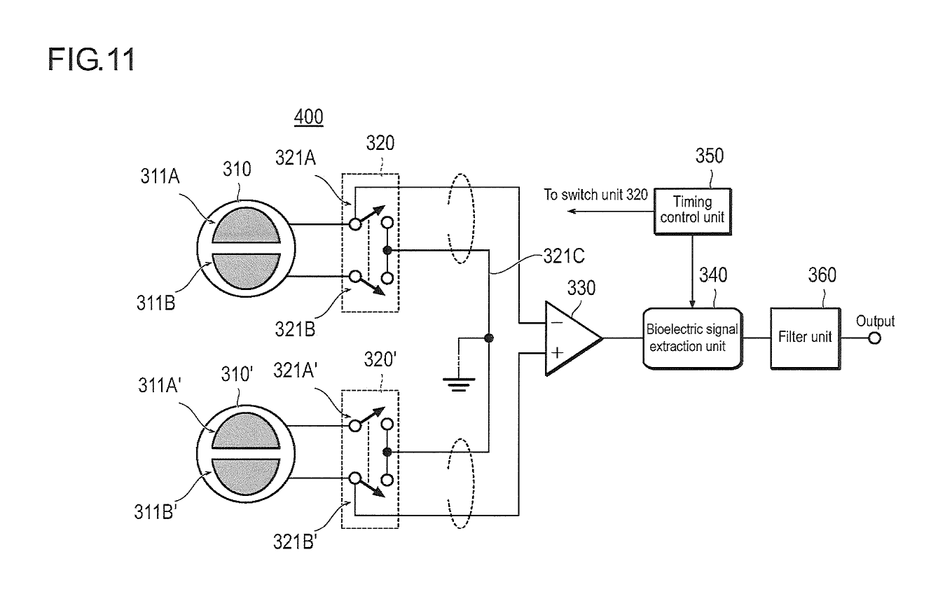

In the first embodiment, a pair of biomedical electrodes has been placed on a living body surface. In the fifth embodiment, two pairs of biomedical electrodes are placed on a living body surface and a bioelectric signal is extracted using the two pairs of biomedical electrodes. With reference to FIG. 11, the bioelectric signal measurement apparatus of the present embodiment will be described below. FIG. 11 is a schematic block diagram for illustrating a configuration of a bioelectric signal measurement apparatus in the fifth embodiment of the present invention.

As illustrated in FIG. 11, a bioelectric signal measurement apparatus 400 of the present embodiment includes first and second biomedical electrode units 310 and 310′, first and second switch units 320 and 320′, a differential amplification unit 330, a bioelectric signal extraction unit 340, a timing control unit 350, and a filter unit 360.

In the present embodiment, configurations except for the first and second biomedical electrode units 310 and 310′ and the switch units 320 and 320′ are the same as in the first embodiment. Therefore, description of configurations other than the configurations of the first and second biomedical electrode units 310 and 310′ and the first and second switch units 320 and 320′ will be omitted.

<First and Second Biomedical Electrode Units>

The first biomedical electrode unit 310 includes biomedical electrodes 311A and 311B, and the second biomedical electrode unit 310′ includes biomedical electrodes 311A′ and 311B′. In the present embodiment, the first biomedical electrode unit 310 and the second biomedical electrode unit 310′ are disposed with a distance longer than the distances between the biomedical electrodes 311A and 311B and between the biomedical electrodes 311A′ and 311B′. The structures of the biomedical electrodes 311A, 311B, 311A′, and 311B′ are the same as in the biomedical electrodes 11A and 11B of the first embodiment and therefore detailed description of the above structures will be omitted.

<First and Second Switch Units>

The first and second switch units 320 and 320′ short-circuit the biomedical electrodes 311A and 311B, and 311A′ and 311B′, respectively. The first switch unit 320 includes analog switches 321A and 321B, and the second switch unit 320′ includes analog switches 321A′ and 321B′.

The biomedical electrode 311A is connected to one terminal of the analog switch 321A and one input terminal (inverting input) of the differential amplification unit 330, and the biomedical electrode 311B is connected to one terminal of the analog switch 321B. Further, the biomedical electrode 311A′ is connected to one terminal of the analog switch 321A′, and the biomedical electrode 311B′ is connected to one terminal of the analog switch 321B′ and the other input terminal (noninverting input) of the differential amplification unit 330. The other terminals of the analog switches 321A, 321B, 321A′, and 321B′ are connected to a jumper line 321C as a short-circuit resistor. The jumper line 321C is connectable to the ground. Note that, a resistance of the jumper line 321C is very small.

In the present embodiment, the analog switches 321A, 321B, 321A′, and 321B′ are switched on or off in response to the first control signal from the timing control unit 350. When the analog switches 321A, 321B, 321A′, and 321B′ are switched on, then the biomedical electrodes 311A, 311B, 311A′, and 311B′ are short-circuited through the jumper line 321C. Thereafter, when the analog switches 321A, 321B, 321A′, and 321B′ are switched off, then short-circuit between biomedical electrodes 311A, 311B, 311A′, and 311B′ is released.

The bioelectric signal measurement apparatus 300 of the present embodiment configured as mentioned above has the following operation.

In the present embodiment, a polarization potential difference is offset between two pairs of biomedical electrodes 311A and 311B, and 311A′ and 311B′ placed on a living body surface, and then using the biomedical electrodes 311A and 311B′ of the two pairs of the biomedical electrodes, a bioelectric signal is extracted.

As mentioned above, the present embodiment having been described produces the following effect in addition to the effects of the first to fourth embodiments.

(f) The bioelectric signal measurement apparatus of the present embodiment offsets a polarization potential using two pairs of biomedical electrodes placed on a living body surface. Therefore, with inhibition of noise mixed into a bioelectric signal, a polarization potential difference between respective biomedical electrodes is able to be effectively canceled. As a result, an SN ratio characteristics of the bioelectric signal is enhanced.

### Sixth Embodiment

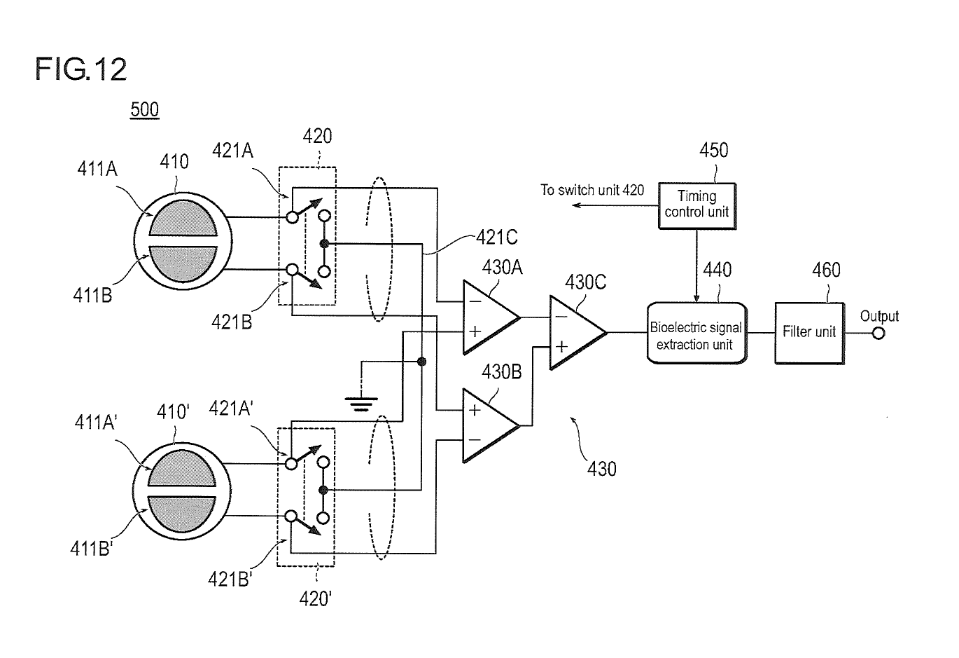

In the fifth embodiment, one biomedical electrode of a pair of biomedical electrodes has been connected to the differential amplification unit to extract a bioelectric signal. In the sixth embodiment, all the biomedical electrodes of two pairs of biomedical electrodes are connected to the differential amplification unit to extract a bioelectric signal. With reference to FIG. 12, the bioelectric signal measurement apparatus of the present embodiment will be described below. FIG. 12 is a schematic block diagram for illustrating a configuration of the bioelectric signal measurement apparatus of the sixth embodiment of the present invention.

As illustrated in FIG. 12, a bioelectric signal measurement apparatus 500 of the present embodiment includes first and second biomedical electrode units 410 and 410′, first and second switch units 420 and 420′, a differential amplification unit 430, a bioelectric signal extraction unit 440, a timing control unit 450, and a filter unit 460.

The present embodiment has the same configuration as in the fifth embodiment except for the connection relationship between the first and second biomedical electrode units 410 and 410′and the first and second switch units 420 and 420′, respectively, and the configuration of the differential amplification unit 430. Therefore, description of configurations other than the connection relationship between the first and second biomedical electrode units 410 and 410′ and the first and second switch units 420 and 420′ and the configuration of the differential amplification unit 430 will be omitted.

The first biomedical electrode unit 410 includes biomedical electrodes 411A and 411B, and the second biomedical electrode unit 410′ includes biomedical electrodes 411A′ and 411B′. In the present embodiment, the first biomedical electrode unit 410 and the second biomedical electrode unit 410′ are disposed with a distance longer than the distances between the biomedical electrodes 411A and 411B and between the biomedical electrodes 411A′ and 411B′. The structures of the biomedical electrodes 411A, 411B, 411A′, and 411B′ are the same as in the biomedical electrodes 11A and 11B of the first embodiment and therefore detailed description of the above structures will be omitted.

The first and second switch units 420 and 420′ short-circuit the biomedical electrodes 411A and 411B, and 411A′ and 411B′, respectively. The first switch unit 420 includes analog switches 421A and 421B, and the second switch unit 420′ includes analog switches 421A′ and 421B′.

<Differential Amplification Unit>

The differential amplification unit 430 includes a first differential amplifier 430A, a second differential amplifier 430B, and a third differential amplifier 430C. An output terminal of the first differential amplifier 430A is connected to one input terminal (inverting input) of the third differential amplifier 430C, and an output terminal of the second differential amplifier 430B is connected to the other input terminal (noninverting input) of the third differential amplifier 430C.

The biomedical electrode 411A is connected to one terminal of the analog switch 421A and one input terminal (inverting input) of the first differential amplification unit 430A, and the biomedical electrode 411B is connected to one terminal of the analog switch 421B and one input terminal (noninverting input) of the second differential amplification unit 430B. Further, the biomedical electrode 411A′ is connected to one terminal of the analog switch 421A′ and the other terminal (noninverting input) of the first differential amplification unit 430A, and the biomedical electrode 411B′ is connected to one terminal of the analog switch 421B′ and the other input terminal (inverting input) of the second differential amplification unit 430B. The other terminals of the analog switches 421A, 421B, 421A′, and 421B′ are connected to a jumper line 421C as a short-circuit resistor. The jumper line 421C is connectable to the ground. Note that, a resistance of the jumper line 421C is very small.

In the present embodiment, the analog switches 421A, 421B, 421A′, and 421B′ are switched on or off in response to the first control signal from the timing control unit 450. When the analog switches 421A, 421B, 421A′, and 421B′ are switched on, then the biomedical electrodes 411A, 411B, 411A′, and 411B′ are short-circuited through the jumper line 421C. Thereafter, when the analog switches 421A, 421B, 421A′, and 421B′ are switched off, then short-circuit between the biomedical electrodes 411A, 411B, 411A′, and 411B′ is released.

The bioelectric signal measurement apparatus 500 of the present embodiment configured as mentioned above has the following operation.

In the present embodiment, a polarization potential difference is an offset between two pairs of biomedical electrodes 411A and 411B, and 411A′ and 411B′ placed on a living body surface, and then using all of the two pairs of the biomedical electrodes, a bioelectric signal is extracted.

As mentioned above, the present embodiment having been described produces the following effect in addition to the effects of the first to fifth embodiments.

(g) The bioelectric signal measurement apparatus of the present embodiment cancels a polarization potential using all of the two pairs of the biomedical electrodes placed on a living body surface and then a bioelectric signal is extracted . Therefore, with inhibition of noise mixed into a bioelectric signal, a polarization potential difference between respective biomedical electrodes is able to be effectively cancels. As a result, an SN ratio characteristics of a bioelectric signal is enhanced.

As mentioned above, in the embodiments, the bioelectric signal measurement apparatus of the present invention has been described. However, it goes without saying that those skilled in the art can make additions to the present invention, modify, and/or omit the present invention appropriately within the scope of the technical ideas of the present invention.

For example, in the first to sixth embodiments, a pair or two pairs of biomedical electrodes have been used. However, in the bioelectric signal measurement apparatus of the present invention, at least three pairs of biomedical electrodes are also employable.

Further, in the first to sixth embodiments, biomedical electrodes placed on a living body surface have been used to measure a bioelectric signal. However, the present invention is not limited to the case where biomedical electrodes are placed on a living body surface to measure a bioelectric signal. For example, the present invention is also applicable to cases in which biomedical electrodes implanted in the cranial nerves, muscles, secreting glands, and others are used to measure a bioelectric signal.

Further, in the first to sixth embodiments, the bioelectric signal measurement apparatus has measured a bioelectric signal continuously from measurement initiation. However, the bioelectric signal measurement apparatus of the present invention is also able to measure a bioelectric signal intermittently. For example, in order to measure an abnormal action potential which may be generated in myocardial ischemia and others, it is possible to continuously measure an electrocardiogram using the bioelectric signal measurement apparatus of the present invention and in addition, to measure a potential in a T-P Interval (time interval between a T wave and a P wave on the electrocardiogram) having no potential at normal time.

Further, in the third embodiment, time constants τ_{G} and τ_{F} have been calculated inside the bioelectric signal measurement apparatus. However, it is possible to calculate the time constants τ_{G} and τ_{F} outside the bioelectric signal measurement apparatus and then to incorporate the calculation results into the bioelectric signal measurement apparatus.

Further, in the third embodiment, four biomedical electrodes have been used. However, it is also possible to use at least five biomedical electrodes, with no limitation to four as the number of biomedical electrodes.

In the first and second embodiments, description has been made for the case of the biomedical electrodes 11A and 11B having the same impedance. In this manner, when biomedical electrodes in which charge-discharge characteristics are the same and electrochemical characteristics are uniform in the biomedical electrodes 11A and 11B are used, an alternate current component and a direct current component of a bioelectric signal are able to be accurately obtained.

This application is based on Japanese Patent Application No. 2011-042935 filed on Feb. 28, 2011, the disclosed contents of which are incorporated entirely herein by reference.

### REFERENCE SIGNS LIST

10: Biomedical electrode unit
11A, 11B: Biomedical electrode
20: Switch unit
21A, 21B: Analog switch
21C: Jumper line
30: Differential amplification unit
40: Bioelectric signal extraction unit
50: Timing control unit
60: Filter unit
100: Bioelectric signal measurement apparatus

1-11. (canceled)

12. A bioelectric signal measurement apparatus comprising:
a plurality of biomedical electrodes in contact with a living body surface disposed separately from each other;
a switch unit for short-circuiting the biomedical electrodes via a predetermined short-circuit resistor;
a differential amplification unit for amplifying a difference of electric signals from the biomedical electrodes, the differential amplification unit being connected to the biomedical electrodes;
a bioelectric signal extraction unit for extracting a bioelectric signal from an output signal of the differential amplification unit during a period of time from the release of short-circuit between the biomedical electrodes to the next timing of short-circuiting; and
a timing control unit for controlling timing for short-circuiting the biomedical electrodes and releasing the short-circuit therebetween, which are repeated.

13. The bioelectric signal measurement apparatus according to claim 12, wherein the bioelectric signal extraction unit extracts the bioelectric signal based on a pulse waveform of an output signal of the differential amplification unit.

14. The bioelectric signal measurement apparatus according to claim 13, wherein the bioelectric signal extraction unit extracts the bioelectric signal from the output signal of the differential amplification unit by approximating the pulse waveform using the following monoexponential regression expression:
y=a₁+b₁·(1−exp(−t/τ))
where y represents an amplitude value of a pulse waveform; a₁ represents a compensation value in calculation; b₁ represents a sum of a biological potential and an electrode potential; and τ represents a pseudo-time constant, where a time constant for a biological potential to charge an electric capacitance between a living body and each biomedical electrode such as an electric double layer and a time constant until a polarization potential reaches an equilibrium state are totaled.

15. The bioelectric signal measurement apparatus according to claim 13, wherein the bioelectric signal extraction unit extracts the bioelectric signal from the output signal of the differential amplification unit by approximating the pulse waveform using the following double exponential regression expression:
y=a₂+b₂·(1−exp(−t/τ))+c·(1−exp(−t/τ₂))
where y represents an amplitude value of a pulse waveform; a₂ represents a compensation value in calculation; b₂ represents a biological potential; τ₁ represents a time constant for a biological potential to charge an electric capacitance between a living body and each biomedical electrode such as an electric double layer; c represents an electrode potential; and τ₂ represents a time constant until a polarization potential reaches an equilibrium state.

16. The bioelectric signal measurement apparatus according to claim 15, wherein processing is carried out by changing a body resistance between the biomedical electrodes with respect to the short-circuit resistance specified, extrapolating a time constant of a pulse waveform output from the differential amplification unit, and calculating a time constant τ_{G} when the body resistance is 0 is carried out with changes in the short-circuit resistance; the time constant τ_{G} for each short-circuit resistance is extrapolated to calculate a time constant τ_{F} when the short-circuit resistance is 0; and the τ_{F} is applied to the τ₂.

17. The bioelectric signal measurement apparatus according to claim 16, wherein a time constant of a pulse waveform output from differential amplification unit having different bias current is extrapolated to calculate a time constant when the bias current is 0; and the time constant is applied to the τ₂.

18. The bioelectric signal measurement apparatus according to claim 13, wherein after a predetermined elapsed time from the release of short-circuit between the biomedical electrodes, an amplitude value of a pulse waveform of the output signal of the differential amplification unit is held and the bioelectric signal is extracted,

19. The bioelectric signal measurement apparatus according to claim 12, wherein the plurality of biomedical electrodes are juxtaposed inside a housing.

20. The bioelectric signal measurement apparatus according to claim 12, wherein the plurality of biomedical electrodes are disposed on concentric circles inside a housing.

21. The bioelectric signal measurement apparatus according to claim 15, wherein when the body resistance per unit distance is known, a pair of the biomedical electrodes are disposed separately with a predetermined distance with respect to the short-circuit resistance specified; and processing is carried out by measuring a time constant of a pulse waveform output from the differential amplification unit and calculating a time constant τ_{G} when the body resistance is 0 is carried out with changes in the short-circuit resistance; the time constant τ_{G} for each short-circuit resistance is extrapolated to calculate a time constant τ_{F} when the short-circuit resistance is 0; and the τ_{F} is applied to the τ₂.

22. The bioelectric signal measurement apparatus according to claim 15, wherein when a pair of the biomedical electrodes are disposed close to each other with making no contact with respect to the short-circuit resistance specified, processing is carried out by measuring a time constant of a pulse waveform output from the differential amplification unit and designating a time constant when the body resistance is 0 as τ_{G} is carried out with changes in the short-circuit resistance; the time constant τ_{G} for each short-circuit resistance is extrapolated to calculate a time constant τ_{F} when the short-circuit resistance is 0; and the τ_{F} is applied to the τ₂.
